(19) United States
(12) Patent Application Publication
(10) Pub. No.: US 2009/0042974 A1
(43) Pub. Date: Feb. 12, 2009

(54) Compositions And Methods Relating To Extensible Transgenic Vector Assembler, Pestilence Ridder, Plus Cannabinoid Producer

(76) Inventors: Anson David Parker, Charlottesville, VA (US); Virginia Lee Valentine-Coles, Charlottesville, VA (US)

Correspondence Address: Mr. Anson Parker, Apt 15, 101 Carrollton Ter., Charlottesville, VA, 22903, US

(21) Appl. No.: 11/835,051

(22) Filed: Aug. 7, 2007

(51) Int. Cl. A01N 43/16; A01P 1/00; C12P 17/06

(52) U.S. Cl. 514/454; 435/125

(57) ABSTRACT

The following provisional patent application refers to the methods and compositions relating to a novel use for enzymatic catalysis of C21-H30-O2 (delta-nine tetrahydrocannabinol (THC)) as an insect repellent, bactericide, and fungicide and dispensation methods as commercial reagent.

The bio-synthesis of cannabinoids represents a landmark achievement in the field of composting, vector removal and ecological reconstitution. Although the benefits have been known for millennia, the advent of modern bio-engineering techniques brings these small seeds of native wisdom to bear on a broader and more industrialized scale—removing dangerous molds and pestilences such as mosquitoes from swamped and flooded regions, raw sewage areas, and disaster sites where ensuing vermin and harmful vectors may cause greater damage than the initial catastrophes.

It is the ambition and intention of authors that tactical usage of this broad-sweeping technique may rapidly and at low cost satisfy a global demand in what may be termed a “grass-roots” bio-engineering project worthy of the 3^{rd} Millennium; bringing to fruition micro-mass-productivity. Should a clear error be found either in spirit or in factual evidence presented please do not hesitate to contact me.

### FIELD OF THE INVENTION

The present invention relates the compositions and methods for controlling harmful pestilence in toilets and sewage, using modular extensible genetic techniques, and the facilitation of rapid, low-cost, cannabinoid production.

### BACKGROUND OF THE INVENTION AND RELATED ARTS

Regions of standing wastewater harboring high concentrations of unprocessed, unfiltered rubbage and manure can be natural sites for disease. The need for low-impact, low-maintenance composting solutions is needed to address city sewers and streets around the world that routinely overflow with toxic bilge. Controlling pests in such regions may often require the use of manufactured chemicals—created along costly and inefficient energy gradients (USPTO U.S. Pat. No. 5,227,537). Many of these chemical treatments present long-term hazards to the environment in the forms of run-off contamination, and build-up. Once introduced into a system these non-biodegradable inorganic compounds may not easily be eradicated Although these methods may be suitable for certain bilge water, especially in treatment facilities where build-up and run-off are not concerns, they do not address the needs of farmers dealing with compost piles, nor sewage water running rampant through the streets of cities that have been obliterated by tsunamis, hurricanes, or tornadoes—wherein there may be an immediate need for rapid decomposition and pestilence ridding. Nor do these prior arts address the need for low impact conversion and or transmutation of toxins and pestilence, nor do these methods establish within the bilge an ecological breeding ground wherein one skilled in the arts might hope to use the nutrient rich, albeit highly toxic, solution for the establishment of biological byproduct. On the other hand, allowing the degradation of the toxic bilge to take place naturally may not be a viable option—as the aforementioned pestilence may soon make a bid to use the nutrient source as a home. In the proposed invention one skilled in the arts would safely apply numerous vectors to convert the bilge into valuable natural resources while simultaneously defending the region from harmful pestilence both through the creation of anti-microbial substances and through direct competition for resources—in much the same way that acidophilus in yogurt out—competes other microbes.

With regards to the issue of treating filth dispersed deep in underground sewers and inaccessible areas—the invention makes a stark contrast to previous arts. Whereas most chemical reactions must obey the laws of Brownian motion or undergo energetically unfavorable processes such as pumping (USPTO U.S. Pat. No. 5,360,556) or heating (USPTO U.S. Pat. No. 6,753,536), enzymatic reactions enabled in motile vectors hold a decisive advantage as they can move through a liquid medium more easily. As one skilled in the arts appreciates the possibility of using a motile plant vector such as the sperm of the gingko would allow even greater motility for the vector. In the preferred embodiment of this invention catalysts hosted in transgenic e. coli, transgenic tobacco root hair, and used in modular extensible vectors controlling the synthesis of compounds such as tetrahydrocannabinolic acid (THCA), cannabigerolic acid (CBGA), cannabichromenic acid (CBMA), the associated long term costs of pestilence control may be reduced dramatically—while simultaneously enriching the soil with valuable nutrients for commercial crops. As one skilled in the arts will appreciate—the long term application of the proposed invention will manifest itself in stages—much as any great culture ranging from ancient cheese and yogurt cultures to present day bio-engineered vectors, each application of the invention may, in the spirit of evolution, lead to a unique bio-transformation specifically adapted to its environment. The proposed invention brings to the table a base level of safer transmutation of certain toxic fungi (Llewellyn 1977), (Turner 1981), infectious microbes, (Van Klingeren 1976), (Schmitz 1973), and insect pests, (Quaghebeur, 1981) as well as infectious disease transmitted through insects such as West Nile Virus (McPartland, 1993). The nature of this invention is energetically favorable, easily propagated, and low up-keep in cost making it also ideal for third-world implementation in the pursuit of cleaner, safer land. In cases of emergency the preferred embodiment might also serve as a possible source of the neuroprotectant delta-nine tetrahydrocannabinol (THC) through the application of heat such as sunlight or direct flame. In the event of a terrorist attack of neurotoxins, for instance, one might as a means of last resort set fire to the growth medium to convert THCA to THC—which upon inhaling provides neuroprotection (Hampson 1998) (Van der Stelt 2001) (Mechoulam 2001) (USPTO U.S. Pat. No. 6,630,507). Whereas in prior arts Elsohy et al (USPTO U.S. Pat. No. 6,730,519) disclosed a method for reduced cost THC production they also rely on traditional abiotic, inorganic, energetically unfavorable means for THC extraction and purification of THC. Moreover their claims depend on natural growth of Cannabis Sativa, a process that may take up to fifteen weeks. Clearly this is not an acceptable waiting period in the case of a terrorist attack. In an alternate embodiment of the invention a serum of raw nutrients, as opposed to raw sewage, were used as the basic medium—in this case using modularized transgenic enzymatic techniques one skilled in the arts might produce several tons of THC in two to three days.

### SUMMARY OF THE INVENTION—OBJECTS

The term “Transgenic Stilbene-carboxylate synthase-like enzyme (TSCSL)” (see Fellermeier 1998) refers to any enzymatic reaction that yields Olivetolic Acid. The trigger mechanism. In alternate embodiments of this invention it is linked operably to a bioluminescent and equipped with a unique “off switch.”

The term “Transgenic Geranylpyrophosphate Prenylase (TOAP)” refers to any enzymatic reaction that yields Cannabigerol (see Fellermeier 1998). In an alternate embodiment linked operably to a bioluminescent and equipped with a unique “off switch”.

The term “Transgenic Cannabigerolic Acid Synthase (TCAs)” (See Raharjo 2002) refers to any enzymatic reaction or nano-bot that synthesizes Cannabigerolic Acid. In alternate embodiments of this invention it is linked operably to a bioluminescent and equipped with a unique “off switch.”

The term “Transgenic Cannabidiolic Acid Synthase (TCBAs)” (see Taura F. 1996) refers to any enzymatic reaction that synthesizes Cannabidiolic Acid. In the preferred embodiment of this invention it is linked operably to a bioluminescent and equipped with a unique “off switch.”

The term “Transgenic Tetrahydrocannabinolic Acid Synthase (TTAs)” refers to any enzymatic reaction that synthesizes THCA, (see reference Taura 2004). In an alternate embodiment of this invention it is linked operably to a bioluminescent and equipped with a unique “off switch.”

The term “Transgenic Cannabichromene Synthase (TCBMs). In an alternate embodiment of this invention it is linked operably to a bioluminescent and equipped with a unique “off switch.” Such bioluminescent switch might include prior arts described in USPTO U.S. Pat. No. 6,544,729, although one skilled in the arts might determine others more suitable.

Genetic “Off switch”—any of several dozen enzymes with known lethality targeting specifically the aforementioned transgenic vectors—each with its own unique off switch. Including but in no way limited to switches described in USPTO U.S. Pat. No. 5,328,847.

The terms “wastewater, raw sewage, bilge water, manure, compost, toxic sludge, filth, festering rot, crud, crude, rubbage, and debris” refers to any medium that may need pestilence management.

The term “pestilence management” refers to the control—be it through repellence, extermination, or slowing of growth rate, of any or several of the following organisms Alabama argillacea (Riley 1885), Pieris brassicae (Beling 1932), Melolontha melolontha (Mateeva 1995), and Aphelenchoides composticola, (Grewal 1989), potato beetle (Leptinotarsa decemlineata) (Stratii 1976), mosquito larvae (Anopheles and Culex species)(Jalees et al. 1993), Chilo partellus, (a lepidopteran borer)(Bajpai and Sharma, 1992), Tetranychus urticae (Fenili and Pegazzano, 1974). Japanese beetles (Metzger and Grant, 1932), Heterodera cajani (Mojumder et al. 1989), Ustilago species (Misra and Dixit 1979, Singh and Pathak 1984), Neovossia indica (Gupta and Singh 1983), Curvularia (Upandhyaya and Gupta, 1989), Colletotrichum truncatum (Kaushal and Paul, 1989), Aspergillus, Penicillium, Cladosporium, Drechslera, Fusarium, Cephalosporium, Rhizopus, Mucor and Curvularia (Pandey, 1982), gram (+) S. aureus, Bacillus megaterium (Veliky and Genest 1972), gram (+) Corynebacterium species and gram (−) Pseudomonas and Agrobacterium species (Bel'tyukova 1962), Trypanosoma brucei (Nok et al., 1994), Phomopsis ganjae (Charles and Jenkins 1914, McPartland 1983), Arctia caja (Rothschild et al., 1977) or any other known or unknown organism with undesirable trails.

The term “transgenically enhanced vector” (TEV) refers to any vector, its parental lineage or its offspring that has been modified by the use of modern or Mendelian genetic techniques to produce a compound.

The term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

Floatation system—in the preferred embodiment floating systems with roots embedded are used to suspend the transgenic roots as they convert cannabigerolic acid into cannabinoids.

The term “bioluminescent protein” refers to a protein capable of causing the emission of light through the catalysis of a chemical reaction. The term includes proteins that catalyze bioluminescent or chemiluminescent reactions, such as those causing the oxidation of luciferins. The term “bioluminescent protein” includes not only bioluminescent proteins that occur naturally, but also mutants that exhibit altered spectral or physical properties.

The term “transformed” refers to a cell into which (or into an ancestor of which) has been introduced, by means of recombinant nucleic acid techniques, a heterologous nucleic acid molecule.

The term “transgenic” is used to describe an organism that includes exogenous genetic material within all of its cells. The term includes any organism whose genome has been altered by in vitro manipulation of the early embryo or fertilized egg or by any transgenic technology to induce a specific gene knockout.

The term “transgene” refers any piece of DNA which is inserted by artifice into a cell, and becomes part of the genome of the organism (i.e., either stably integrated or as a stable extrachromosomal element) which develops from that cell. Such a transgene may include a gene which is partly or entirely heterologous (i.e., foreign) to the transgenic organism, or may represent a gene homologous to an endogenous gene of the organism. Included within this definition is a transgene created by the providing of an RNA sequence that is transcribed into DNA and then incorporated into the genome. The transgenes of the invention include DNA sequences that encode the fluorescent or bioluminescent protein that may be expressed in a transgenic non-human animal, the genes required for the synthesis of cannabinoids, and any additional genetic information necessary for the greater control of the invention.

The following terms are used to describe the sequence relationships between two or more polynucleotides: “reference sequence”, “comparison window”, “sequence identity”, “percentage identical to a sequence”, and “substantial identity”. A “reference sequence” is a defined sequence used as a basis for a sequence comparison; a reference sequence may be a subset of a larger sequence, for example, as a segment of a full-length cDNA or gene sequence, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Natl. Acad. Sci. (U.S.A.) 85:2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage identical to a sequence” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 30 percent sequence identity, preferably at least 50 to 60 percent sequence identity, more usually at least 60 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. As applied to polypeptides, the term “substantial identity” means that two peptide sequences, when optimally aligned, such as by the programs GAP or BESTFIT using default gap weights, share at least 30 percent sequence identity, preferably at least 40 percent sequence identity, more preferably at least 50 percent sequence identity, and most preferably at least 60 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

Since the list of technical and scientific terms cannot be all encompassing, any undefined terms shall be construed to have the same meaning as is commonly understood by one of skill in the art to which this invention belongs. Furthermore, the singular forms “a”, “an” and “the” include plural referents unless the context clearly dictates otherwise. For example, reference to a “restriction enzyme” or a “high fidelity enzyme” may include mixtures of such enzymes and any other enzymes fitting the stated criteria, or reference to the method includes reference to one or more methods for obtaining cDNA sequences which will be known to those skilled in the art or will become known to them upon reading this specification.

### SUMMARY OF THE INVENTION—OPERATION

As one skilled in the arts may appreciate the variety of vectors able to transform of the initial reagents (TSCSL, TOAP, TCAs, TTAs, TCBMs, TCBAs) into the desired reagents (THC, CBD, CBM) may result in hundreds or thousands of potential scenarios. Consider the heat that is generated in many compost conversion where temperatures may rise above 160 degrees Fahrenheit, in such cases it may be expedient to use an thermophilic vector, particularly for the incubation of the TSCSL, and TOAP. In the preferred embodiment of the invention it should be noted that the TTAs, TCBMs, TCBAs, are used in either a plant or animal vector—since cannabinoids exhibits both anti-microbial and anti-fungal activity it will require a non-microbial and non-fungal host.

In its preferred embodiment begin with a gigantic pile of refuse, that may include fecal matter, untreated sewage water, and decaying animal parts. It may be to the advantage of the user to initiate the enzymatic activity in a more sterile environment with nutrients needed for the synthesis of precursors of cannabinoids to alleviate environmental pressures of the sludge. In such cases as necessary the resulting enzymes and precursor products may be added directly to the filthy sludge or set aside and used as the growth medium for the transgenic cannabinoid synthesis with the resulting cannabinoids added to the filth sludge after their synthesis is completed.

Expose the pile of refuse to TSCSLs, TOAPs, and TCs teas—brewed as per the guideline in the literature commonly as anyone skilled in the arts will appreciate—and genetically modified to include promoters operably linked to bioluminescent proteins to help indicate and monitor effectiveness of the treatment. This tea is given from 24 hours to one month as indicated by bioluminescence (FIG. 1) to finish blending in with the refuse—or as long as the bioluminescence appears active. (FIG. 1. Step: Stilbene-like Synthase). These teas, when mixed with toxic bilge, enzymatically synthesize cannabinoids.

Next a root bed made of TTAs, TCBAs, and or TCBMs. Note the versatility of this invention. Any one of the aforementioned synthases, or indeed all three may be placed atop the pile bilge to create the desired reagents (ie THC, CBA, CBM). Also noteworthy is the elegant closed-loop nature of this system. By initiating the reaction with microbes that are not themselves immune to the final product the system will eventually turn itself off—as the reagent levels rise to higher levels the TSCSLs, TOAPs, and TCs die.

### IN AN ALTERNATE EMBODIMENT

The bucket containing the OAP is loosened atop a pile of crud, that may consist of any decaying or decayed matter, and that must consist of some decaying vegetable matter or living vegetation.

The bucket containing the CAS is loosened atop the pile of crud that previously received OAP treatment.

A blanket of roots from tobacco made of TTAs, TCBAs and or TCBMs are thrown over the crapulence and festering therein may it yield bountifully wee little cannabinoids.

### Overview

This invention relates to the synthesis of cannabinoids for the purpose of general pestilence riddance in filthy organic and inorganic sludge. Through regulated enzymatic reactions, wherein cannabinoids with known anti-microbial, insecticidal, nematicidal, fungicidal properties and moreover nutritious, and neuroprotective, qualities are used to benefit regions where other commercial chemical reagents would require mechanized dispersion and cleanup. In plain English for those skilled in the arts—the genes involved in the enzymatic formulation of cannabinoids are inserted into foreign vectors thereby reproducing themselves and generating sufficient quantities of cannabinoids to clear the region of pestilence.

The advantages of this system are numerous. Whereas cannabinoid synthesis may not easily take place in Cannabis sativa due to its illegality, this invention is highly preferable. Whereas cannabinoid synthesis using inorganic techniques is not advantageous due to the inefficiency of inorganic and organic laboratory chemistry, this invention is highly preferable. Whereas most chemical synthesis routes for the creation of cannabinoids relate to the creation of extremely pure cannabinoids, this invention merely creates sufficient quantities as needed to rid a region of pestilence, and makes no claims whatsoever as to purity. Whereas the cost of creating cannabinoids synthetically would require large sums of money, as well as recurring costs for reagents, as well as a high degree of expertise and lab equipment, the invention described herein requires a single up-front cost to create the necessary vectors, and thereafter the invention may be distributed and applied to sludge and filth across the world with almost no requirements insofar a priori knowledge.

Using closed-loop modular enzymatic reactions, wherein each phase of catalysis may be halted by another counter reaction, and wherein each phase of catalysis may be easily monitored for effectiveness allows one skilled in the arts to more safely and effectively treat hazardous waste and the plethora of contagions therein. This invention refers to modular, in the sense that along the enzymatic pathway of choice each enzymatic building block is separated into a unique vector, uniquely identifiable by means bioluminescence and uniquely susceptible to a flavor of anti-microbial or anti-fungal such that the enzymatic process of choice may be halted at any given phase of production if desired. Modular may also or rather refer to the system as a whole, in that it should, handled by one skilled in the arts, leave little or no trace of enzymatically active reagent and be a closed-loop system—with the understanding that in nature there exists no such thing as an entirely closed-loop system, however, the preferred embodiment of this invention has in its design constructs a self-destruct or self-neutralizing mechanism for the living reagents. Thus in the preferred embodiment of the invention the catalysis of tetrahydrocannabinolic acid results in the recursive destruction of the initial vectors (Taura 2004).

### DESCRIPTION

Polyketide Synthesis converts 3 Malonyl CoA plus 1 n-Hexanoyl-CoA to form OSCoA. This conversion may take place inside the muck and sludge, or may take place in a contained area and after the OSCoA

Stilbene Carboxylate Synthase-Like (STCSL), in the case of Cannabis Sativa a Chalcone Synthase (CHS) that exhibits Stilbene Synthase (STS) activity in vivo and as per note in the literature (Raharjo 2004) there is reason to believe that the sequis used in the preferred embodiment of this invention and refers to any enzyme that generates 5-amylresorcinolic acid (olivetolic acid). While there are several enzymes capable of synthesizing olivetolic acid in the final analysis any enzyme capable of Olivetolic Acid synthesis will is sufficient. In the preferred embodiment the STCSL is inserted into the mitochondrial genome using the protofection technique (Khan 2004). The STCSL should be operably linked to bioluminescent protein to facilitate the monitoring of activity. The vector of the STCSL should also, in the preferred embodiment, have an operably linked In an alternate embodiment of this invention olivetolic acid is synthesized through inorganic techniques and thus added to the filthy sludge as a trigger molecule. In this manner the invention would have a limiting reagent from the offset, restricting the final output of pestilence ridders in such cases wherein limitations might be preferable. In another alternate embodiment of the invention the STCSL is chimeric with GOAP, or a pestilence ridding molecule.

Geranylpyrophosphate Olivetolic Acid Prenylase (GOAP) (Fellermeier 1998) is an integral part of this invention, and converts olivetolic acid into cannabigerolic acid. As one skilled in the art may appreciate any enzyme capable of yielding cannabigerolic acid is sufficient. In the preferred embodiment the GOAP is loaded into the vector in the manner described in Fellermeier's work. In the preferred embodiment of this invention the GOAP is operably linked to a bioluminescent protein such as GFP or aequorin, and thus its activation is more easily monitored with minimal technical expertise. The GOAP is also operably linked to a promoter capable of up-regulating GOAP and thereby amplifying GOAP production.

Products made from these transgenic vectors should produce THCA, and, in addition, other precursor molecules as well as the necessary enzymes and proteins requisite for the aforementioned production, such as, tetrahydrocannibigerolic acid synthase, cannabigerolic acid synthase (CBGAS), cannabidiolic acid synthase (CBDAS), cannabichromenic acid synthase (CBRMAS), tetrahydrocannibinolic acid (THCA), olivetolic acid, polyketide synthase, and cannabigerolic acid synthase. Also disclosed is the unique and novel application of the TTAs in the function of a compost toilet additive and for the low-impact, sustainable, macrobiotic control of pests including Alabama argillacea (Riley 1885), Pieris brassicae (Beling 1932), Melolontha melolontha (Mateeva 1995), and Aphelenchoides composticola, (Grewal 1989).

### Operation

First the transgenically enhanced vectors (TEVs) as necessary and leading up to the cannabigerolic acid phase of biosynthesis (FIG. 1) are added into the growth medium and let to rest for anywhere from 12 hours to several days with a temperature range of 25-35 degrees centigrade, and also depending on the volume of waste, the thickness of the muck, and the general nature of the festering filth. If time is of the essence one may speed up growth times by dispersing units of TEVs around the afflicted region through artificial or assisted means. If precision in timing is desired it may be convenient to include a bioluminescent protein operably linked a functional promoter to the TEVs similar in methods to (USPTO U.S. Pat. No. 6,544,729) and created such as to reflect the activity of the TEVs.

Next the transgenic plant vector is placed atop the festering sludge. The transgenic plant vector releases cannabinoids into the sludge, and as it appropriates greater the product of transgenic E. Coli(s) so shall it release cannabinoids—all the while eradicating both the transgenic E. Coli vector as well as the numerous pathogens, microbes, insects, fungi etc . . . that are defenseless against the cannabinoids.

### BRIEF DESCRIPTION OF THE DRAWINGS

Many of the attendant advantages of the invention become more readily apparent as the same become better understood by reference to the following detailed description, which taken with the accompanying drawing.

FIG. 1 provides a basic visual understanding for one skilled in the arts to process enzymatic THC, a drawing of the 3 phases of reaction.

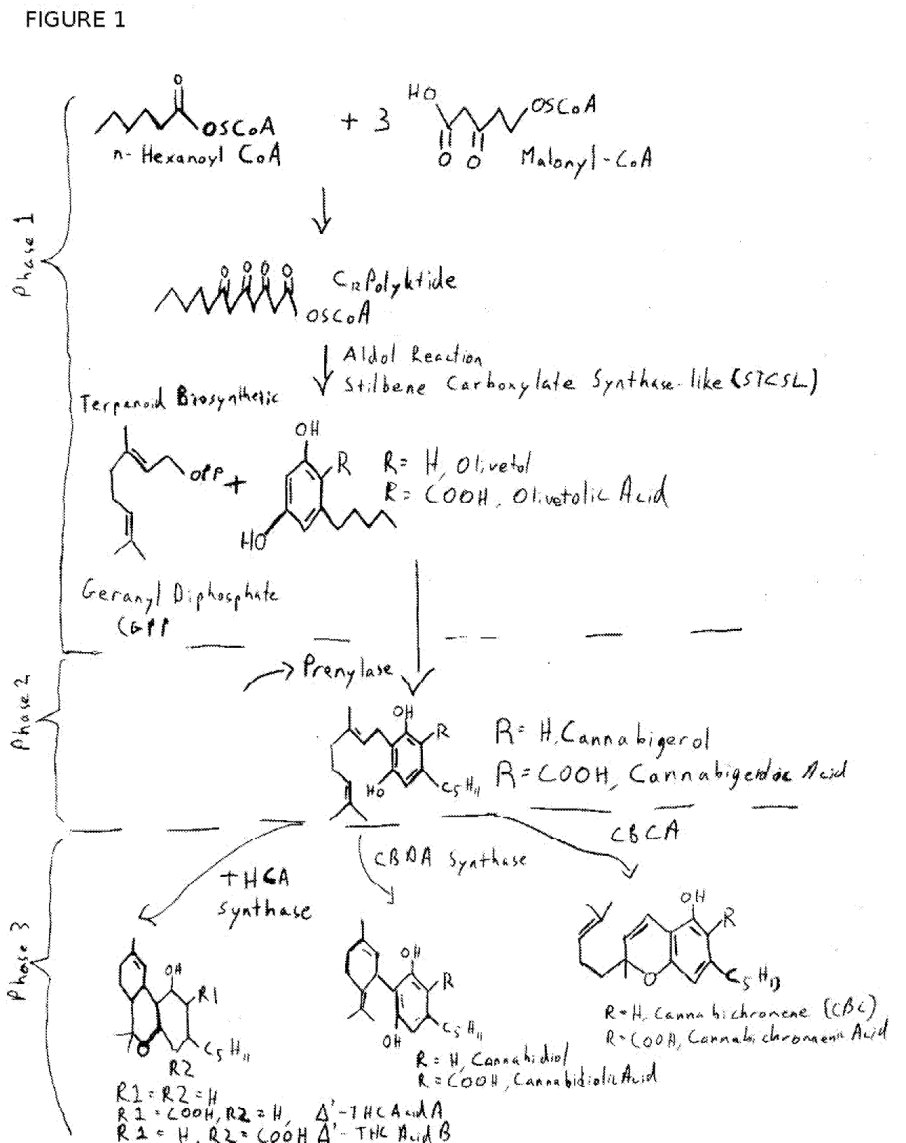

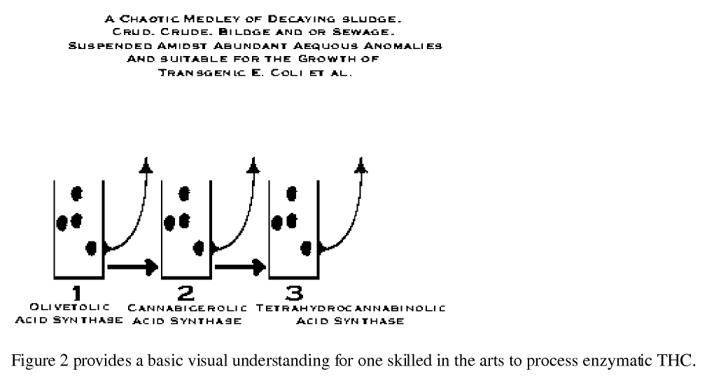

TABLE 1

| Enzyme | Reagents | Product | Time | Temp | Vector | Key References | Accession # |
|---|---|---|---|---|---|---|---|
| Polyketide | 3 MalonylCo-A | OSCoA | 1-24 hrs | 25-35 C. | E. Coli M15 | Raharjo, 2004 | AY082343 |
| STCSL/ | & 1 n-Hexanoyl- | Olivetolic Acid | | | | | |
| CHS | CoA | | | | | | |
| | OSCoA | | | | | | |
| CBDAs | Cannabigerolic | Cannabichromenic | 24-48 hrs | 25-35 C. | Tobacco | Morimoto, 1999 | |
| | Acid | Acid | | | Root Hairs | | |
| Prenylase | Olivetolic Acid + GPP | Cannabigerolic Acid | 1-24 hrs | 25-35 C. | E. Coli M15 | Fellermeier, 1998 | |
| CBCAs | Cannabigerolic | Cannabidiolic Acid | 24-48 hrs | 25-35 C. | Tobacco | Morimoto, 1999 | |
| | Acid | | | | Root Hairs | | |
| THCAs | Cannabigerolic | Tetrahydrocannabinolic | 24-48 hrs | 25-35 C. | Tobacco | Taura, 1995 & | AB057805 |
| | Acid | Acid | | | Root Hairs | Sirikantaramas 2004 | |

### REFERENCES IN THE US PATENT OFFICE

| Author | Title | USPTO # | Keyword | Date |
|---|---|---|---|---|
| Grobler, Marius; | Sewage sludge treatment | 20050175516 | Compost | NE |
| et al. | | | | |
| Liang | Shooting mechanism of an | 6,615,815 | Anti-violence | Sep. 9, |
| | anti-violence gun | | | 2003 |
| Becker, et al. | Method and arrangement of | 5,692,446 | Anti-violence | Dec. 2, |
| | equipment for the protection of | | | 1997 |
| | buildings and people from acts | | | |
| | of violence | | | |
| Sun | Apparatus for preventing | 4,811,775 | Anti-violence | Mar. 14, 1989 |
| | criminal's escape or violence | | | |
| Peterson, et al. | SPANN: Sequence processing | 5,067,095 | Modular Vector | Nov. 19, |
| | artificial neural network | | | 1991 |
| Case, et al. | Thin membrane sensor with | 5,328,847 | Modular switch | Jul. 12, 1994 |
| | biochemical switch | | | |
| Humphreys, et | Apparatus for neutralizing | 6,753,536 | Wastewater | Jun. 22, 2004 |
| al. | chemical and biological threats | | cleaning | |
| | | | apparatus | |
| Ball, et al. | Method of feeding wastewater | 5,360,556 | Wastewater | Nov. 1, |
| | effluent to filter bed through | | cleaning | 1994 |
| | parallel conduits | | apparatus | |
| Hampson, et al. | Cannabinoids as antioxidants | 6,630,507 | Cannabis | Oct. 7, |
| | and neuroprotectants | | Neuroprotectant | 2003 |
| Elsohly, et al. | Method of preparing delta-9- | 6,730,519 | THC synthesis | Dec. 4, |
| | tetrahydrocannabinol | | | 2001 |
| Growcock, et al. | Vermiculture compositions | 6,838,082 | compost | Jan. 4, |
| | | | biolumin | 2005 |
| Sayler; Gary S. | Bioluminescent biosensor | 6,544,729 | Bioluminbiosensor | Apr. 8, 2003 |
| | device | | device | |
| Croteau, et al. | Isolation and bacterial | 6,258,602 | cannabis | Jul. 10, 2001 |
| | expression of a sesquiterpene | | insecticide | |
| | synthase cDNA clone from | | | |
| | peppermint (mentha × piperita, L.) | | | |
| | that produces the aphid | | | |
| | alarm phromone (E)-.beta.- | | | |
| | farnesene | | | |
| Goodwin, Neil | Production of delta 9 | 20050171361 | THC synthesis | Aug. 4, 2005 |
| John; et al | tetrahydrocannabinol | | | |
| Martin, Billy R; | Cannabinoids | 20050165259 | Cannabinoids | Jul. 28, 2005 |
| et al. | | | | |
| Moore, Bob M. | Cannabinoid derivatives, | 20040242593 | THC synthesis | Dec. 2, |
| II; et al. | methods of making, and use | | | 2004 |
| | thereof | | | |
| Chowdhury, | Tetrahydrocannabinol | 20040229939 | THC | Nov. 18, |
| Dipak K.; et | compositions and methods of | | manufacture & | 2004 |
| al. | manufacture and use thereof | | use | |
| Webster, et al. | Cannabinoid extraction method | 6,403,126 | Cannabinoid | Jun. 11, 2002 |
| | | | extraction | |
| McKinney | Method and apparatus for | 4,279,824 | THC extraction | Jul. 21, 1981 |
| | processing herbaceous plant | | | |
| | materials including the plant | | | |
| | cannabis | | | |

### REFERENCES IN THE LITERATURE

1. Abe I, Watanabe T, Noguchi H. Enzymatic formation of long-chain polyketide pyrones by plant type III polyketide synthases. Phytochemistry. 2004 September; 65(17):2447-53.
2. Abrol B. K. and I. C. Chopra, 1963. Development of indigenous vegetable insecticides and insect repellents. Bulletin Jamu Regional Res. Lab 1:156. 1963.
3. Bajpai N. K. and V. K. Sharma. Possible use of hemp (Cannabis sativa L.) weeds in integrated control. Indian Farmers' Digest 25(12):32, 38, 1992
4. Beling I. Schädlingsbekämpfung im 18. Jarhhundert. Anz. Schädlingbekämpfung 8(6):66-69., 1932.
5. Business Alliance for Commerce in Hemp, “Hemp: Friend to People and Ecology” Los Angeles, Calif., April 1994
6. Cekmecelioglu D, Demirci A, Graves R E., Feedstock optimization of in-vessel food waste composting systems for inactivation of pathogenic microorganisms., J Food Prot. 2005 March; 68(3):589-96.
7. Chopra R. N., R. L. Badhwar and S. L. Nayar, 1941. Insecticidal and piscicidal plants of India. J. Bombay Nat. Hist. Soc. 42:854-902.
8. Dahiya M. S. and G. C. Jain, 1977. Inhibitory effects of cannabidiol and tetrahydrocannabinol against some soil inhabiting fungi. Indian Drugs 14(4):76-79.
9. Deportes I, Benoit-Guyod J L, Znirou D, Bouvier M C., Microbial disinfection capacity of municipal solid waste (MSW) composting., J Appl Microbiol. 1998 August; 85(2):238-46.
10. Eckermann C, Schroder G, Eckermann S, Strack D, Schmidt J, Schneider B, Schroder J. Stilbenecarboxylate biosynthesis: a new function in the family of chalcone synthase-related proteins. Phytochemistry. 2003 February; 62(3):271-86.
11. Eisenreich, W., Schwarz, M., Cartayrade, A., Arigoni, D., Zenk, M. H. & Bacher, A. (1998) The deoxyxylulose phosphate pathway of terpenoid biosynthesis in plants and microorganisms. Chem. Biol. 5, R221″R233.
12. Fellermeier M, Eisenreich W. Bacher A, Zenk M H., Biosynthesis of cannabinoids. Incorporation experiments with (13)C-labeledglucoses. Eur J Biochem. 2001 March; 268(6):1596-604.
13. Ferenczy L. Antibacterial substances in seeds. Nature 178:639-640., 1956.
14. Ferenczy L., L. Gracza and I. Jakobey. An antibacterial preparatum from hemp (Cannabis sativa). Naturwissenschaften 45:188., 1958.
15. Ferrer J L, Jez J M, Bowman M E, Dixon R A, Noel J P., Structure of chalcone synthase and the molecular basis of plant polyketide biosynthesis. Nat Struct Biol. 1999 August; 6(8):775-84. Bioresour Technol. 2001 December; 80(3):217-25.
16. Gal I. E., O. Vajda and I. Bekes. A kannabidiolsav nehany tulaj-donsaganak vizsgalata élelmiszertartósítási szempontból. Elelmiszervizsgalati Közlemenyek 4:208-216.1969.
17. Grainge M. and S. Ahmed. Handbook of Plants with Pest-Control Properties. John Wiley and Sons, NY. 470 pp., 1988.
18. Grewal P. S. Effects of leaf-matter incorporation on Aphelenchoides composticola (Nematoda), mycofloral composition, mushroom compost quality and yield of Agaricus bisporus. Annals Applied Biology 115:299-312., 1989.
19. Gupta R. P. and A. Singh. Effect of certain plant extracts and chemicals on teliospore germination of Neovossia indica. Indian J. Mycology and Plant Pathology 13(1):116-117. 1983.
20. Hampson A J, Grimaldi M, Axelrod J, Wink D. Cannabidiol and (−)Delta9-tetrahydrocannabinol are neuroprotective antioxidants. Proc Natl Acad Sci USA 1998 Jul. 7; 95(14):8268-73
21. Hassen A, Belguith K, Jedidi N, Chemf A, Chemf M, Boudabous A., Microbial characterization during composting of municipal solid waste.
22. Jager E, Ruden H, Zeschmar-Lahl B., [Composting facilities. 1. Microbiological quality of compost with special regard to disposable diapers], Zentralbl Hyg Umweltmed. 1994 October; 196(3):245-57. German.
23. Jalees S., S. K. Sharma, S. J. Rahman and T Verghese, 1993. Evaluation of insecticidal properties of an indigenous plant, Cannabis sativa L., against mosquito larvae under laboratory conditions. J. Entomol. Res. 17:117-120.1993.
24. Jenkins, Phil, “Field of Opportunity” Canadian Geographic, Mar. 19, 1999
25. Kane V V, Razdan R K. Constituents of hashish. A novel reaction of olivetol with citral in the presence of pyridine. Total synthesis of dl-cannabicyclol and dl-cannabichromene. J Am Chem Soc. 1968 Nov. 6; 90(23):6551-3.
26. Kashyap N. P., R. M. Bhagat, D. C. Sharma and S. M. Suri, 1992. Efficacy of some useful plant leaves for the control of potato tuber moth, Phthorimaea operculella Zell. in stores. J. Entomological Research 16:223-227. 1992.
27. Khan S M, Bennett J P Jr., Development of mitochondrial gene replacement therapy., J Bioenerg Biomembr. 2004 August; 36(4):387-93. Review.
28. Kir'yanova E. S. and E. L. Krall. Plant-Parasitic Nematodes and their Control, Vol. II. Academy of Sciences of the USSR, Nauka Publishers, Leningrad. 1971.
29. Klingeren B. van and M. T. Ham. Antibacterial activity of delta-9-tetrahydrocannabinol and cannabidiol. Antonie van Leeuwenhoek 42:9-12., 1976.
30. Kok C. J., G. C. M. Coenen, and A. de Heij, 1994. The effect of fibre hemp (Cannabis sativa L.) on selected soil-borne pathogens. J. International Hemp Association 1(1):6-9.
31. Kurilov V. I. and N. S. Kakhta. [More about hemp and the Colorado beetle.] Zashchita Rastenii 1977 (7):63. 1977.
32. Lenehan N A, DeRouchey J M, Marston T T, Marchin G L. Concentrations of fecal bacteria and nutrients in soil surrounding round-bale feeding sites., J Anim Sci. 2005 July; 83(7):1673-9.
33. Llewellyn G C, O'Rear C E., Examination of fungal growth and aflatoxin production on marihuana., Mycopathologia. 1977 Dec. 16; 62(2):109-12.
34. Loewe S., 1946. Studies on the pharmacology and acute toxicity of compounds with marihuana activity. J. Pharmacology and Expermental Therapeutics 88:154-164.
35. Mackiewicz S., 1962. The effect of hemp on the density of the potato beetle and the bean aphid. Biul. Ochrona Roslin Inst. 16:101-131.
36. Mateeva A. Use of unfreindly plants against root knot nematodes. Acta Horticulturae 382 (February):178-182. 1995.
37. McPartland J. M., Fungal pathogens of Cannabis sativa in central Illinois. Phytopathology 73:797., 1983,
38. McPartland J. M. Pathogenicity of Phomopsis ganjae on Cannabis sativa and the fungistatic effect of cannabinoids produced by the host. Mycopathologia 87:149-153., 1984
39. McPartland, John M., Cannabis as repellent and pesticide, Journal of the International Hemp Association 4(2): 87-92, 1997
40. Mechoulam R, Hanu L. The cannabinoids: an overview. Therapeutic implications in vomiting and nausea after cancer chemotherapy, in appetite promotion, in multiple sclerosis and in neuroprotection. Pain Res Manag 2001 Summer; 6(2): 67-73.
41. Mechoulam R, Spatz M, Shohami E. Endocannabinoids and neuroprotection. Sci STKE April 23; (129):RE5. 2002.
42. Metzger F. W. and D. H. Grant. Repellency to the Japanese beetle of extracts made from plants immune to attack. USDA Technical Bulletin no. 299. 21 pp., 1932.
43. Misra S. B. and S. N. Dixit. Antifungal activity of leaf extracts of some higher plants. Acta Botanica Indica 7:147-150, 1979.
44. Mojumder V, S. D. Mishra, M. M. Haque and B. K. Goswami. Nematicidal efficacy of some wild plants against pigeon pea cyst nematode, Heterodera cajani. Int. Nematol. Network Newsletter 6(2):21-24, 1989.
45. Nok A. J., S. Ibrahim, S. Arowosafe, et al. The trypanocidal effect of Cannabis sativa constituents in experimental animal try-panosomiasis. Veterinary and Human Toxicology 36:522-524, 1994.
46. S. Morimoto, F. Taura, Y. Shoyama, Biosynthesis of cannabinoids in Cannabis sativa L, Curr. Top. Phytochem. 2 (1999) 103-113.
47. Oku, T. and Katsura, Y “Sequence analysis encoding alpha-helix-turn-alpha-helix motif of HrpX in plant pathogenic Xanthomonas”, Unpublished
48. Pandey K. N. Antifungal activity of some medicinal plants on stored seeds of Eleusine coracana. J. Indian Phytopathology 35:499-501.1982.
49. Pandey J. and S. S. Mishra. Effects of Cannabis sativa L. on yield of rabi maize (Zea mays L.). in Abstracts of papers, Annual conference of Indian Society of Weed Science. Bihar, India. 1982.
50. Prakash A., I. C. Pasalu and K. C. Mathur, 1982. Evaluation of plant products as paddy grain protectants in storage. International J. Entomology 1:75-77.
51. Prakash A., J. Rao and I. C. Pasalu, 1987. Studies on stored grain pests of rice and methods of minimising losses caused by them. Final Project Report (RPF-III) Ent-6/CRRI/ICAR (India). 33 pp.
52. Quaghebeur K, Coosemans J, Toppet S, Compemolle E, Cannabiorci- and 8-chlorocannabiorcichromenic acid as fungal antagonists from Cylindrocarpon olidum., Phytochemistry. 1994 September; 37(1):159-61.
53. Radosevic A., M. Kupinic and L. Grlic. Antibiotic activity of various types of Cannabis resin. Nature 195:1007-1009. 1962.
54. Raharjo, Tri J; Chang, Wen-Te; Verberne, Marianne C; Peltenburg-Looman, Anja M G; Linthorst, Huub J M; Verpoorte, Robert, Cloning and over-expression of a cDNA encoding a polyketide synthase from Cannabis sativa, Plant Physiology and Biochemistry—Paris, 42 (4), 291-298, 2004.
55. Riley C. V. and L. O. Howard. Hemp as a protection against weevils. Insect Life (USDA) 4: 223 1892.
56. Rothschild M., M. R. Rowan and J. W. Fairbairn. Storage of cannabinoids by Arctia caja and Zonocerus elegans fed on chemically distinct strains of Cannabis sativa. Nature 266:650-651. 1977.
57. Schmitz J A, Olson L D., Duration of viability and the growth and expiration rates of group E streptococci in soil., Appl Microbiol. 1973 February; 25(2):180-3.
58. Shoyama, Y., Hirano, H., and Nishioka, I. (1978) J. Labelled Ccmpd. Radiopharm. 14, 835-842
59. Sirikantaramas S, Morimoto S, Shoyama Y, Ishikawa Y, Wada Y, Shoyama Y, Taura F., The gene controlling marijuana psychoactivity: molecular cloning and heterologous expression of Delta1-tetrahydrocannabinolic acid synthase from Cannabis sativa L., J Biol Chem. 2004 Sep. 17; 279(38):39767-74. Epub 2004 June 9.
60. Stratii Y. I. Hemp and the Colorado beetle. Zashchita Rastenii 5:61., 1976.
61. Taura, F., Morimoto, S., Shoyama, Y., and Mechoulam, R. (1995) J. Am. Chem Soc. 117, 9766-9767
62. Taura, F., Morimoto, S., and Shoyama, Y. (1996) J. Biol. Chem. 271, 17411-17416
63. F. Taura, S. Morimoto and Y. Shoyama, Biosynthesis of marihuana compounds—Purification and characterization of biosynthetic enzymes. Current Topic in Plant Biology, Vol. 2 p 63-73 (2000), 2000.
64. Turner C E, Elsohly M A., Biological activity of cannabichromene, its homologs and isomers., J Clin Pharmacol. 1981 August-September; 21(8-9 Suppl):283S-291S.
65. Van der Stelt M, Veldhuis W B, Bar P R, Veldink G A, Vliegentharet J F, Nicolay K. Neuroprotection by Delta9-tetrahydrocannabinol, the main active compound in marijuana, against ouabain-induced in vivo excitotoxicity. J Neurosci 2001 Sep. 1; 21(17): 6475-
66. Van Haute, E., Joos, H., Maes, M., Warren, G., Van Montagu, M., and Schell, J. EMBO J. 2, 411-417, 1983.
67. Van Klingeren B, Ten Ham M. Antibacterial activity of delta9-tetrahydrocannabinol and cannabidiol., Antonie Van Leeuwenhoek; 42(1-2):9-12 1976.
68. Veliky I. A. and R. K. Latte. Antimicrobial activity of cultured plant cells and tissues. Lloydia 37:611-620., 1974.
69. Vijai P., I. Jalali and R. D. Parashar. Suppression of bacterial soft rot of potato by common weed extracts. J. Indian Potato Association 20:206-209, 1993.
70. White, F. F., and Nester, E. W. J. Bacteriol. 141, 1134-1141, 1980.
71. T. Yamaguchi, F. Kurosaki, D.-Y. Suh, U. Sankawa, M. Nishioka, T. Akiyama, M. Shibuya, Y. Ebizuka, Cross-reaction of chalcone synthase and stilbene synthase overexpressed in Escherichia coli, FEBS Lett. 460 457-461, 1999.
72. Zelepukha S. I., 1960. The third conference on the problem of phytoncides. J. Mikrobiol, Kiev 22(1):68-71.A

1. We do hereby claim that enzymatically biosynthesized delta-9-tetrahydrocannabinol and delta-9-tetrahydrocannabinolic-acid provides a novel and unique technique for ridding sludge and sewage containing bacterium, insects and their larvae, fungi, and other non-human organisms. This claim extends to all enzymatically generated cannabinols grown in any non-human organism specifically for the purpose of pestilence killing or minimization, with especial claim to the techniques illustrated in the summary of the invention and Table 1, wherein one skilled in the arts may clearly review the methods for said endeavor.
We do hereby claim that the methods of enzymatically generating delta-9-tetrahydrocannabinol and delta-9-tetrahydrocannabinolic-acid are both unique and novel and of substantial benefit to humanity as a low cost strategy for generating industrial quantities of said compounds.
